(19) United States
(12) Patent Application Publication
(10) Pub. No.: US 2026/0256451 A1
(43) Pub. Date: Sep. 3, 2026

(54) ULTRASONIC PROBE AND ULTRASONIC DEVICE

(71) Applicant: WUHAN UNITED IMAGING HEALTHCARE CO., LTD., Wuhan (CN)

(72) Inventors: Xianglong Ma, Wuhan (CN); Kang Si, Wuhan (CN)

(21) Appl. No.: 19/658,931

(22) Filed: Apr. 27, 2026

Related U.S. Application Data
(63) Continuation-in-part of application No. 18/909,999, filed on Oct. 9, 2024, now Pat. No. 12,611,178.

(30) Foreign Application Priority Data
Nov. 30, 2023 (CN) 202311641396.3

(51) Int. Cl. A61B 8/00; G01S 15/89

(52) U.S. Cl. CPC A61B 8/4494; A61B 8/4444; A61B 8/461; G01S 15/892; G01S 15/8927

(57) ABSTRACT

The present disclosure relates to an ultrasonic probe and an ultrasonic device. The ultrasonic probe includes a first transducer and a second transducer. The first transducer is arranged in a first direction and at least includes a first portion and a second portion; the second transducer is arranged in a second direction, the second direction being perpendicular to the first direction; wherein the second transducer is located between the first portion and the second portion of the first transducer.

### CROSS-REFERENCE TO RELATED APPLICATIONS

This application is a continuation-in-part application of US patent application No. 18/909,999, filed on October 9, 2024, which claims priority to Chinese patent application No. 202311641396.3, filed on November 30, 2023, the contents of each of which are hereby incorporated by reference in their entireties, respectively.

### TECHNICAL FIELD

The present disclosure relates to the field of medical technologies, and in particular, to an ultrasonic probe and an ultrasonic device.

### BACKGROUND

Ultrasonic devices are commonly used to scan an object to be detected (such as a certain part of a patient’s body) for diagnostic imaging.

Ultrasonic probe is an important component of the ultrasonic device. According to different clinical uses, the ultrasonic probes have many different types. An intracavitary probe is an ultrasonic probe that can be inserted into a cavity of a human organ, which can be used for trans-anorectal, trans-vaginal or trans-esophageal examinations, for example.

Taking the application of the probe in intracavitary ultrasound puncture diagnosis as an example, in the related art, during an insertion of the probe into the cavity, a position of a puncture needle and a position of a punctured tissue cannot be simultaneously presented in an ultrasonic image. Doctors need to frequently move the probe to determine the positions of the punctured tissue and the puncture needle, which affects the detection efficiency.

### SUMMARY

Based on this, it is necessary to provide an ultrasonic probe to address the problem of low detection efficiency in conventional ultrasonic probes.

According to a first aspect of the present disclosure, there is provided an ultrasonic probe, which includes a transducer assembly. The transducer assembly includes: a first transducer arranged in a first direction and at least including a first portion and a second portion; and a second transducer arranged in a second direction, the second direction being perpendicular to the first direction; wherein the second transducer is located between the first portion and the second portion of the first transducer.

In an embodiment, the first transducer includes two convex arrays arranged in the first direction, the first portion of the first transducer includes one of the two convex arrays located on a first side in the first direction, and the second portion of the first transducer includes the other of the two convex arrays located on a second side opposite to the first side in the first direction ; and the second transducer includes a linear array arranged in the second direction, and the linear array is arranged between the first portion and the second portion.

In an embodiment, a field of view angle of each of the convex arrays is an obtuse angle.

In an embodiment, the linear array and the two convex arrays are arranged to form a cross shape.

In an embodiment, the linear array extends a first distance relative to the convex arrays in the second direction, and a ratio of the first distance to a length of the linear array is higher than 0 and less than or equal to 2/3.

In an embodiment, the ratio of the first distance to the length of the linear array is higher than or equal to 1/4 and less than or equal to 1/2.

In an embodiment, each of the convex arrays includes a first lens layer, and the first lens layer has a first bonding surface. The linear array includes a second lens layer, and the second lens layer has second bonding surfaces. Each of the second bonding surfaces is connected to one of the first bonding surfaces.

In an embodiment, an orientation of each of the first bonding surfaces is in conformity with an orientation of one of the second bonding surfaces located on a same side.

In an embodiment, the first bonding surfaces and the second bonding surfaces are each a cambered surface.

In an embodiment, along the second direction, a distance between the first bonding surfaces of the two convex arrays gradually decreases first and then gradually increases.

In an embodiment, a minimum distance between the two first bonding surfaces is greater than an elevation of the linear array.

In an embodiment, each of the convex arrays further includes a first piezoelectric layer stacked with the first lens layer in a third direction; each of the convex arrays further includes a first matching layer arranged between the first lens layer and the first piezoelectric layer; each of the convex arrays further includes a first backing layer stacked on a side of the first piezoelectric layer facing away from the first matching layer; the linear array further includes a second piezoelectric layer stacked with the second lens layer in the third direction; the linear array further includes a second matching layer arranged between the second lens layer and the second piezoelectric layer; and the linear array further includes a second backing layer stacked on a side of the second piezoelectric layer facing away from the second matching layer; wherein the third direction, the first direction, and the second direction are perpendicular to each other.

In an embodiment, each of the convex arrays further includes a first heat dissipation member connected to the first backing layer; and the linear array further includes a second heat dissipation member connected to the second backing layer.

In an embodiment, the first backing layer and the first heat dissipation member are provided with a first concave-convex matching portion and a second concave-convex matching portion configured to engage with the first concave-convex matching portion, respectively; and/or the second backing layer and the second heat dissipation member are provided with a third concave-convex matching portion and a fourth concave-convex matching portion configured to engage with the third concave-convex matching portion, respectively.

In an embodiment, the first heat dissipation member is connected to a side of the first backing layer facing away from the first matching layer; and/or the second heat dissipation member is connected to a side of the second backing layer facing away from the second matching layer.

In an embodiment, the first heat dissipation member is connected to a side of the first backing layer in the first direction; and/or the second heat dissipation member is connected to a side of the second backing layer in the first direction.

In an embodiment, the first lens layer and the second lens layer are in an integrated structure; and/or the two first backing layers are in an integrated structure; and/or the two first heat dissipation members are in an integrated structure.

In an embodiment, the ultrasonic probe further includes a connecting member, and the linear array and the convex arrays are each connected to the connecting member.

In an embodiment, the connecting member includes a first support section and second support sections connected to both sides of the first support section in the first direction; the ultrasonic probe further includes a first fastener configured to connect the first support section and the linear array; and the ultrasonic probe further includes second fasteners, each of the second fasteners being configured to connect one of the second support sections and the convex array located on a same side.

In an embodiment, the second support sections are slidably connected to the first support section, and are capable of driving the convex arrays to move in the second direction.

In an embodiment, the first transducer includes two convex arrays arranged in the first direction, the first portion includes one of the two convex arrays located on the first side in the first direction, and the second portion includes the other of the two convex arrays located on the second side opposite to the first side in the first direction; and the second transducer includes a first sub-linear array, a first area array, and a second sub-linear array arranged in the second direction, the first area array is connected between the first sub-linear array and the second sub-linear array, and the first area array is arranged between the first portion and the second portion.

According to a second aspect of the present disclosure, there is provided an ultrasonic probe, which includes a transducer assembly. The transducer assembly includes: a first transducer arranged in a first direction and at least including a first portion and a second portion; and a second transducer arranged in a second direction, the second direction being perpendicular to the first direction; wherein the second transducer is located between the first portion and the second portion of the first transducer, and wherein, in the first direction, a first piezoelectric layer in the first portion of the first transducer and a first piezoelectric layer in the second portion of the first transducer are spaced apart from each other and located on two sides of the second transducer.

According to a third aspect of the present disclosure, there is provided an ultrasonic probe, which includes a transducer assembly. The transducer assembly includes: a first transducer including a second area array arranged in a first direction; and a second transducer including a first sub-linear array and a second sub-linear array arranged in a second direction, the second direction being perpendicular to the first direction; wherein the second area array is located between the first sub-linear array and the second sub-linear array.

According to a fourth aspect of the present disclosure, there is provided an ultrasonic device. The ultrasonic device includes a host, a display, and an ultrasonic probe according to any of the above aspects. The ultrasonic probe is configured to acquire ultrasonic imaging data. The host is communicatively connected with the ultrasonic probe, and is configured to receive and process the ultrasonic imaging data to generate an ultrasonic image. The display is connected to the host, and is configured to display the ultrasonic image.

The details of one or more embodiments of the present disclosure are set forth in the accompanying drawings and the description below. Other features, objects, and advantages of the present disclosure will become apparent from the description, the drawings, and the claims.

### BRIEF DESCRIPTION OF THE DRAWINGS

FIG. 1 is a schematic diagram illustrating an ultrasonic probe according to various embodiments of the present disclosure.

FIG. 2 is a cross-sectional view of the ultrasonic probe shown in FIG. 1.

FIG. 3 is a schematic diagram illustrating a transducer assembly in the ultrasonic probe according to various embodiments of the present disclosure.

FIG. 4 is a schematic diagram of a transducer assembly according to a first embodiment of the present disclosure.

FIG. 5 is a simplified diagram illustrating an imaging area of the transducer assembly shown in FIG. 4.

FIG. 6 is a top view of the transducer assembly shown in FIG. 4.

FIG. 7 is a partial cross-sectional view of the transducer assembly shown in FIG. 4.

FIG. 8 is an exploded view of the transducer assembly shown in FIG. 4.

FIG. 9 is a cross-sectional view of the transducer assembly shown in FIG. 4.

FIG. 10 is a cross-sectional view of a transducer assembly according to another example of the first embodiment of the present disclosure.

FIG. 11 is a schematic diagram illustrating a transducer assembly according to another example of the first embodiment of the present disclosure.

FIG. 12 is a schematic diagram illustrating a transducer assembly according to another example of the first embodiment of the present disclosure.

FIG. 13 is a schematic diagram illustrating a transducer assembly according to another example of the first embodiment of the present disclosure.

FIG. 14 is a schematic diagram illustrating a transducer assembly according to another example of the first embodiment of the present disclosure.

FIG. 15 is a schematic diagram of a transducer assembly according to a second embodiment of the present disclosure.

FIG. 16 is a schematic diagram of the transducer assembly in FIG. 15 with transparent layers omitted.

FIG. 17 is a top view of the transducer assembly shown in FIG. 16.

FIG. 18 is a partial cross-sectional view of the transducer assembly shown in FIG. 16.

FIG. 19 is a schematic diagram of a transducer assembly according to a third embodiment of the present disclosure.

FIG. 20 is a schematic diagram of the transducer assembly in FIG. 19 with transparent layers omitted.

FIG. 21 is a top view of the transducer assembly shown in FIG. 19.

FIG. 22 is a schematic diagram of the transducer assembly shown in FIG. 21 with transparent layers omitted.

FIG. 23 is a partial cross-sectional view of the transducer assembly shown in FIG. 19.

### Reference numbers:

10, ultrasonic probe; 11, transducer assembly; 1, first transducer; 2, second transducer; 100, convex array; 101, FOV boundary; 110, first lens layer; 111, first bonding surface; 120, first piezoelectric layer; 130, first matching layer; 140, first backing layer; 141, first concave-convex matching portion; 150, first heat dissipation member; 151, second concave-convex matching portion; 160, first flexible printed circuit layer; 200, linear array; 201, first sub-linear array; 202, second sub-linear array; 210, second lens layer; 211, second bonding surface; 220, second piezoelectric layer; 230, second matching layer; 240, second backing layer; 241, third concave-convex matching portion; 250, second heat dissipation member; 251, fourth concave-convex matching portion; 260, second flexible printed circuit layer; 300, connecting member; 310, first support section; 311, first screw hole; 320, second support section; 321, second screw hole; 410, shell acoustic head end; 420, shell handle end; 430, electrical connection lead; 440, cable; 450, mainboard; 500, first area array; 501, second area array; 510, third lens layer; 520, third piezoelectric layer; 530, third matching layer; 540, third backing layer; 550, third heat dissipation member; 560, third flexible printed circuit layer; 570, chip layer.

### DETAILED DESCRIPTION OF THE EMBODIMENTS

In order to make the above objectives, features and advantages of the present disclosure more obvious and understandable, specific implementations of the present disclosure are described in detail below with reference to the accompanying drawings. In the following description, many specific details are set forth in order to fully understand the present disclosure. However, the present disclosure can be implemented in many other ways different from those described herein, and those skilled in the art can make similar improvements without departing from the connotation of the present disclosure. Therefore, the present disclosure is not limited by the specific embodiments disclosed below.

In the description of the present disclosure, it should be understood that if the terms “center”, “longitudinal”, “transverse”, “length”, “width”, “thickness”, “upper”, “lower”, “front”, “back”, “left”, “right”, “vertical”, “horizontal”, “top”, “bottom”, “inner”, “outer”, “clockwise”, “counterclockwise”, “axial”, “radial”, “circumferential”, etc. are used, these terms indicate the orientation or position relationship as shown in the accompanying drawings and are merely intended to facilitate the description of the present disclosure and simplify the description, rather than indicating or implying that the indicated device or element must have a specific orientation or be constructed and operated in a specific orientation. Therefore, these terms are not to be interpreted as limiting the present disclosure.

In addition, if the terms such as “first” and “second” are used, they are used for descriptive purposes only, and should not be understood as indicating or implying relative importance or implicitly indicating the quantity of the technical features indicated. Thus, the features described with “first” and “second”, etc., may explicitly or implicitly include at least one of these features. In the description of the present disclosure, if the term “plurality” is used, it means at least two, such as two, three, etc., unless otherwise clearly and specifically defined.

In the present disclosure, unless otherwise clearly specified and limited, if the terms “mounted”, “coupling”, “connection”, “fixation”, etc., are used, these terms should be understood in a broad sense, for example, it may be a fixed connection, a detachable connection, or integration. It may be a mechanical connection or an electrical connection. It may be a direct connection or an indirect connection through an intermediate medium. It may be an internal connection between two array elements or an interaction relationship between the two array elements, unless otherwise clearly defined. Those of ordinary skill in the art can understand the specific meanings of the above terms in the present disclosure according to specific situations.

In the present disclosure, unless otherwise clearly specified and limited, if there is a description that a first feature is “on” or “under” a second feature, etc., it may mean that the first and second features are in direct contact, or the first and second features are in indirect contact through an intermediate medium. Moreover, the first feature being “on”, “above”, or “over” the second feature may mean that the first feature is directly above or obliquely above the second feature, or simply means that the horizontal height of the first feature is greater than that of the second feature. The first feature being “under”, “beneath” and “below” the second feature may mean that the first feature is directly below or obliquely below the second feature, or simply means that the horizontal height of the first feature is less than the that of second feature.

It should be noted that when an element is referred to as being “fixed on” or “arranged on” another element, it may be directly on the other element or there may be an intervening element. When an element is referred to as being “connected to” another element, it may be directly connected to the other element or there may also be an intermedium element. If present, the terms “vertical”, “horizontal”, “upper”, “lower”, “left”, “right” and similar expressions used in this disclosure are for the purpose of illustration only and are not meant to be the only implementation methods.

As used herein, the terms "substantially," "generally," "approximately," "about," and similar relative terms are intended to have a broad meaning in harmony with the common and accepted usage by those of ordinary skill in the art to which the subject matter of this disclosure pertains. These terms are used herein to describe and claim various features, and are not intended to be strictly numerical or geometric in nature. Rather, these terms are intended to allow for some variation from the stated or ideal condition, as may be reasonably acceptable in view of the relevant art, manufacturing tolerances, and the intended function of the feature being described. It should be understood that these terms are descriptive terms commonly used in patent claims to avoid a strict numerical boundary to a specified parameter, and the scope of such terms should be interpreted in light of the teachings of the specification and the understanding of one of ordinary skill in the art. Unless otherwise specified, a deviation of up to ±10% (or any other percentage or range as may be appropriate for the particular context) from a stated value or condition shall be considered within the scope of such relative terms. In the absence of a specific numerical standard, the scope of these terms shall be determined by whether the feature in question performs its intended function in substantially the same way to achieve substantially the same result.

In the related art, an ultrasonic probe is generally consisted of two transducer arrays, including a linear array for longitudinal plane imaging and a convex array for transverse plane imaging. The two transducer arrays are generally arranged in a T-shape, resulting in a blind area in an imaging area. In a case that positions of a punctured tissue and a puncture needle cannot be simultaneously presented in an ultrasonic image, doctors need to frequently move the probe to determine the positions of the punctured tissue and the puncture needle, which affects the detection efficiency.

Based on this, the present disclosure provides an ultrasonic probe 10 that can solve at least one of the above problems.

For ease of description, the ultrasonic probe 10 provided by the present disclosure may be defined with a first direction, a second direction, and a third direction that are orthogonal to each other. The first direction is the X direction shown in the figures(which may also be referred to as the left-right direction), the second direction is the Y direction shown in the figures(which may also be referred to as the front-rear direction), and the third direction is the Z direction shown in the figures(which may also be referred to as the up-down direction).

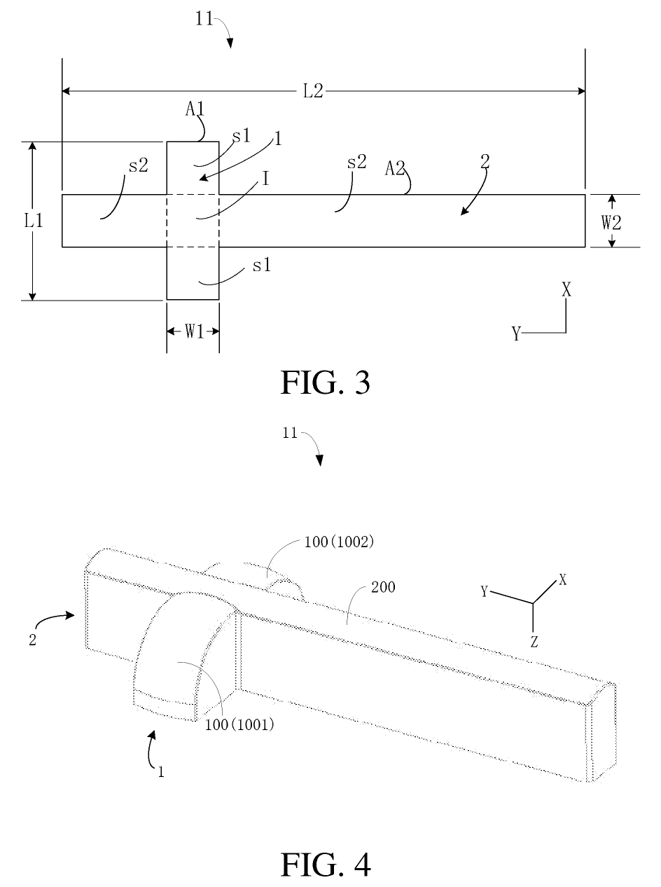

The ultrasonic probe 10 provided by the present disclosure may include a transducer assembly 11, and the transducer assembly 11 may (e.g., generally) include a first transducer 1 and a second transducer 2. Referring to FIG. 3, when viewed from top to bottom along the Z direction, the first transducer 1 is (e.g., generally) arranged in a first rectangular area A1, which has a first length L1 in the first direction and a first width W1 in the second direction. When viewed from top to bottom along the Z direction, the second transducer 2 is (e.g., generally) arranged in a second rectangular area A2, which has a second length L2 in the second direction and a second width W2 in the first direction. Thus, the length direction of the first transducer 1 and the length direction of the second transducer are perpendicular to each other. Wherein, the first transducer 1 and the second transducer 2 intersect at an intersection to form an intersection area I in the ultrasonic probe 10 (see the area indicated by the dashed box in FIG. 3), such that the ultrasonic probe 10 formed by combining the first transducer 1 and the second transducer 2 is (e.g., generally) arranged in a cross shape. Correspondingly, for ease of description, the first rectangular area A1 can be divided into the intersection area I and two first sub-areas s1 located on both sides of the intersection area I in the first direction. Similarly, the second rectangular area A2 can be divided into the intersection area I and two second sub-areas s2 located on both sides of the intersection area I in the second direction. In some embodiments, the second length L2 of the second transducer 2 may be longer than the first length L1 of the first transducer 1 to adapt to the required shape of the detection end of the ultrasonic probe 10 (for example, see the shell acoustic head end 410 in FIG. 1). Accordingly, the first transducer 1 is configured to perform sagittal plane imaging, and the second transducer 2 is configured to perform coronal plane imaging. In some embodiments, the first transducer 1 is configured to perform sagittal plane imaging, and the second transducer 2 is configured to perform coronal plane imaging simultaneously.

The first transducer 1 may be at least arranged in the two first sub-areas s1, and the second transducer 2 may be at least arranged in the two second sub-areas s2. In addition, only a part of components of the first transducer 1 may be arranged in the intersection area I, or only a part of components of the second transducer 2 may be arranged in the intersection area I, a combination of both a part of components of the first transducer 1 and a part of components of the second transducer 2 may be arranged in the intersection area I, or an additional component of the first transducer 1 and the second transducer 2. In the various exemplary embodiments to be specifically described below, by appropriately arranging the configurations of the first transducer 1 and/or the second transducer 2 in the intersection area I, as well as the structures, shapes, etc. of the first transducer 1 and/or the second transducer 2 themselves, and by enabling the first transducer 1 and the second transducer 2 being configured to perform imaging detection simultaneously (i.e., transmit and receive ultrasonic waves simultaneously), cross-imaging of two planes, the sagittal plane and the coronal plane, can be achieved in the intersection area I, thereby enabling real-time scanning of information of the two planes of the same body part.

The transducer assembly 11 of the ultrasonic probe 10 in various embodiments of the present disclosure will be described in detail below with reference to the accompanying drawings.

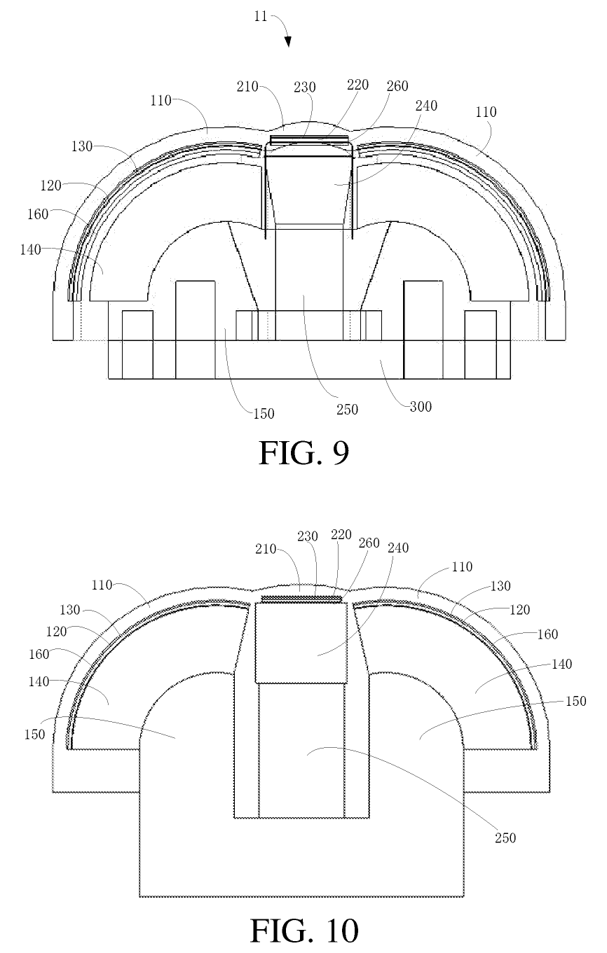

Referring to FIGS. 4 to 14, a transducer assembly 11 according to a first embodiment of the present disclosure is provided. The transducer assembly 11 provided in the first embodiment of the present disclosure includes a first transducer 1 and a second transducer 2. In this first embodiment, the second transducer 2 may be arranged in the two second sub-areas s2 and the intersection area I. The first transducer 1 is at least partially arranged in the two first sub-areas s1, and at least a part of components in the first transducer 1 are separated by the second transducer 2. In addition, optionally, some components in the first transducer 1 may also extend into the intersection area I.

In the first embodiment, the first transducer 1 is (e.g., generally) arranged in the first direction and at least includes a first portion 1001 and a second portion 1002 spaced apart along the first direction, and the second transducer 2 is located between the first portion 1001 and the second portion 1002 of the first transducer 1, so that the first transducer 1 and the second transducer 2 are combined to form a cross-shaped structure.

Specifically, referring to FIGS. 4 to 8, the first transducer 1 includes at least two convex arrays 100 arranged in the first direction, and the linear array 200 is located between at least a part of components of the two convex arrays 100. Specifically, the first portion 1001 may include the left convex array 100, the second portion 1002 may include the right convex array 100, and the linear array 200 is located between the first portion 1001 and the second portion 1002. In some application scenarios, the arrangement direction of the linear array 200 is the forward scanning direction of the ultrasonic probe 10.

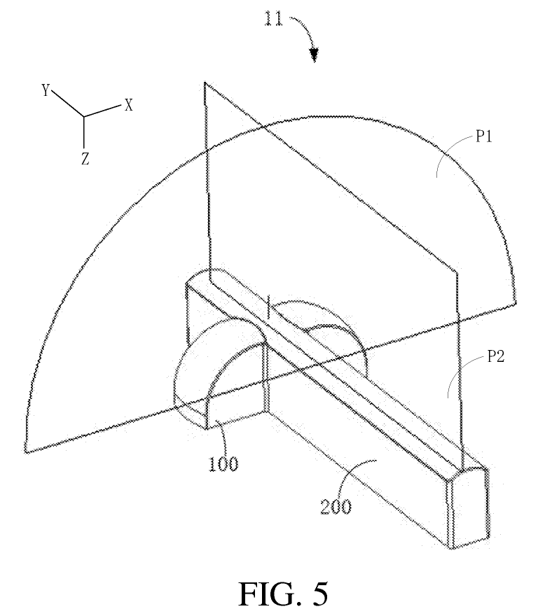

The convex arrays 100 are configured to perform coronal plane imaging and the linear array 200 is configured to perform sagittal plane imaging. By performing imaging using both the convex arrays 100 and the linear array 200 simultaneously, information on the two planes of one same body part can be scanned (e.g., in real time). Specifically, each of the convex arrays 100 can transmit and receive ultrasonic waves in its own radial direction. After the ultrasonic waves received by the convex arrays 100 are received and processed by a host of an ultrasonic device, an ultrasonic image of a radial area surrounding an outer peripheral surface of the probe may be formed. For example, the convex arrays 100 can detect an inner wall of a cavity and tissues close to the inner wall of the cavity to obtain a coronal plane image, so that a position of a punctured tissue can be observed. Referring to FIG. 5, a two-dimensional coronal plane imaging plane P1 for detection by the convex arrays 100 is shown, which is perpendicular to the second direction. The linear array 200 can transmit and receive ultrasonic waves to and from an outer peripheral wall of the ultrasonic probe 10. After the ultrasonic waves received by the linear array 200 are received and processed by the host of the ultrasonic device, an ultrasonic image of a front end area is formed. For example, the linear array 200 can detect the position of the puncture needle to obtain a sagittal plane image. Referring to FIG. 5, a two-dimensional sagittal plane imaging plane P2 for detection by the linear array 200 is shown, which is perpendicular to the first direction. During use, the convex arrays 100 and the linear array 200 transmit and receive ultrasonic waves simultaneously, so that the positions of the punctured tissue and the tip of the puncture needle can be observed simultaneously, without the need for frequently moving the ultrasonic probe 10 to switch between two planes, thereby accurately achieving the intracavitary puncture operation and improving the detection accuracy and efficiency.

It should be noted that the above first transducer may be consisted of two independent convex arrays 100, and the two convex arrays 100 are not connected to each other. That is, the two convex arrays 100 are arranged spaced apart from each other in the first direction. Alternatively, a middle part of one convex array may be processed to form a trench in the middle part, and the trench is configured to connect the linear array, such that said one convex array forms an irregular-shaped convex array, and two ends of the irregular-shaped convex array in the first direction form the two convex arrays 100 included in the first transducer as described above. That is, the two convex arrays 100 may also be connected to each other to form as an integrated structure, for example, via an intermediate structure(s) connected between the two convex arrays 100 and bypassing below the linear array 200. The specific configurations can be selected according to actual needs, which is not limited here.

In an embodiment, the ultrasonic probe 10 of the embodiments of the present disclosure can be applicable to intracavitary detection of various organs, for example, trans-esophageal, trans-vaginal and trans-anal examinations, but is not limited thereto. The ultrasonic probe 10 may also be used to inspect incisions formed on the body, which is not limited here.

In an embodiment, the convex arrays 100 for coronal plane imaging may be made of an array transducer with a plurality of array elements and a preset frequency range, for example, made of an array transducer with 80 array elements and a frequency of 3 MHz to 10 MHz. The array transducer forming the convex arrays 100 may be referred to as a convex array transducer. In the convex array transducer, a distance between two array elements may be within a range of 0.13 mm to 0.2 mm, and an elevation, i.e., a width of a positive end of the array element, is within a range of 5 mm to 6 mm, and a curvature radius of each of the convex arrays 100 may be within a range of 7 mm to 8.5 mm. The linear array 200 for sagittal plane imaging may be made of an array transducer with a plurality of array elements and a preset frequency range, for example, made of an array transducer with 256 array elements and a frequency of 3 MHz to 14 MHz. The array transducer forming the linear array 200 may be referred to as a linear array transducer. In the linear array transducer, a distance between two array elements may be within a range of 0.2 mm to 0.25 mm, and an elevation is within a range of 3 mm to 5 mm. The convex array transducer and the linear array transducer may each be a 1.5D array or a 1.75D array, etc. In other embodiments, the convex array transducer and the linear array transducer may also each have 96 array elements or 192 array elements, etc. Taking a row of array elements in the transducer as an example, an arrangement direction of the plurality of array elements of the linear array 200 is the same as a length direction of the linear array 200, and an arrangement direction of the plurality of array elements of the convex arrays 100 is perpendicular to the arrangement direction of the plurality of array elements of the linear array 200. In other embodiments, two rows of array elements, a plurality of rows of array elements, etc. may also be arranged in the transducer. The number of array elements of the convex array transducer and the number of array elements of the linear array transducer may be determined according to the size and imaging requirement of the convex arrays 100 and the size and imaging requirement of the linear array 200. The preset frequency range of the convex array transducer and the preset frequency range of the linear array transducer may be determined according to usage needs.

In this first embodiment, illustratively, the arrangement of the array elements in the transducer may be a one-dimensional array (1D Array) in which a plurality of array elements are arranged in a row along a line, and the one-dimensional array can scan at the same time to obtain a two-dimensional slice image in a two-dimensional plane defined by the arrangement direction of the row of array elements and the detection direction of the row of array elements. It should be noted that the one-dimensional array described in the present disclosure may include, for example, 1D, 1.25D, 1.5D and other generalized one-dimensional arrays known in the art, as long as the array elements are generally arranged along a line to detect two-dimensional images. Taking

the arrangement of a row of array elements in the transducer to form a one-dimensional array as an example, the arrangement direction of the plurality of array elements of the linear array 200 is the same as the length direction of the linear array 200, i.e., the array elements are arranged in a row along a straight line extending in the second direction, thereby forming a one-dimensional linear array (1D linear array). The arrangement direction of the plurality of array elements of the convex arrays 100 is perpendicular to the arrangement direction of the plurality of array elements of the linear array 200, and the array elements can be arranged in a curved manner along the bending direction of the convex arrays 100, i.e., arranged in a row along an arc extending along the bending direction of the convex arrays 100 in a plane perpendicular to the second direction, thereby forming a one-dimensional convex array (1D convex array). The two convex arrays 100 on the left and right sides each form a one-dimensional convex array, and the adjacent ends of the one-dimensional convex arrays of the two convex arrays 100 are aligned with each other in the first direction (but spaced apart by the linear array 200), so that the array elements of the left convex array 100 and the array elements of the right convex array 100 generally still form a combined one-dimensional convex array arranged on an arc extending along the overall bending direction of the two convex arrays 100 in a plane perpendicular to the second direction. In this way, the imaging area of the combined one-dimensional convex array formed by the plurality of array elements of the two convex arrays 100 generally falls within the aforementioned coronal plane imaging plane P1 in FIG. 5, wherein the left convex array 100 forms a roughly fan-shaped two-dimensional imaging area P11 (an area within the FOV of the left convex array 100 in the coronal plane imaging plane P1), and the right convex array 100 forms a roughly fan-shaped two-dimensional imaging area P12 (an area within the FOV of the right convex array 100 in the coronal plane imaging plane P1), see FIGS. 12, 13, and 14. The imaging areas P11 and P12 are both within the coronal plane imaging plane P1, and the two combine to form the overall imaging area

of the two convex arrays 100. The imaging area of the one-dimensional linear array formed by the plurality of array elements of the linear array 200 also falls within the aforementioned sagittal plane imaging plane P2 in FIG. 5 (e.g., the same as the aforementioned sagittal plane imaging plane P2), and the imaging area of the linear array 200 may be a roughly rectangular two-dimensional imaging area above the one-dimensional linear array. In other embodiments, two rows of array elements, multiple rows of array elements, etc., each row arranged in a one-dimensional array as above, may also be arranged in the transducer. Wherein, the number of array elements in the convex array transducer and the number of array elements in the linear array transducer may be appropriately determined according to, for example, the imaging requirements and sizes of the convex arrays 100 and the linear array 200. The preset frequency range of the convex array transducer and the preset frequency range of the linear array transducer may be determined according to usage needs.

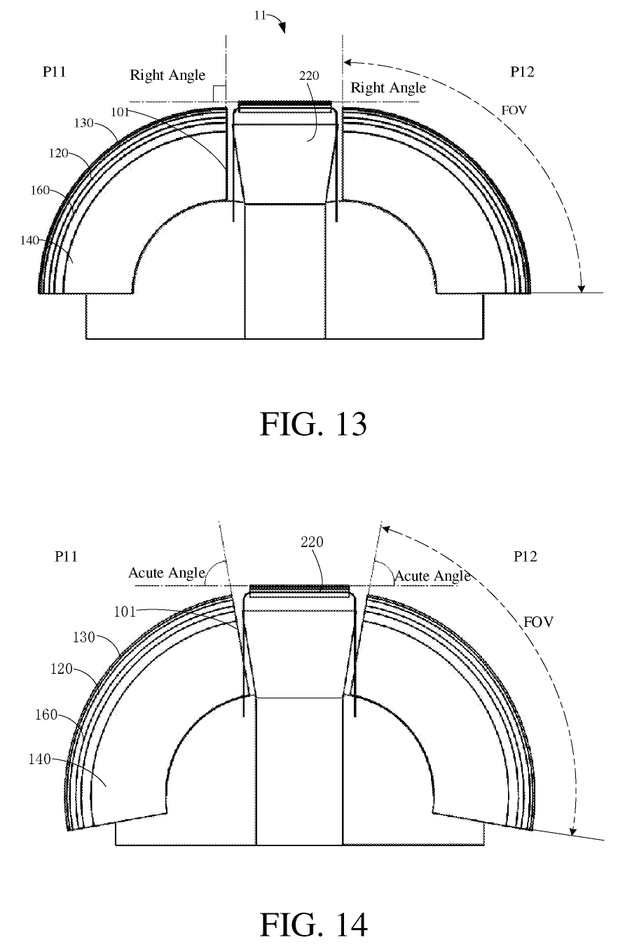

As shown in FIG. 12, in some embodiments, a field of view (FOV) angle of each of the convex arrays 100 is an obtuse angle. The field of view angle is an angle between a plane where the second piezoelectric layer 220 of the linear array 200 is located and a FOV boundary 101 of the convex array 100, and the boundary 101 refers to a lateral side of the convex array 100 facing the linear array 200. Through this arrangement, it is ensured that the imaging plane P1 (e.g., including the imaging areas P11 and P12) of the convex arrays 100 and the imaging plane P2 of the linear array 200 have a sufficient intersection area therebetween. Furthermore, the reliability of simultaneously observing the positions of the punctured tissue and the tip of the puncture needle through the intersection area is ensured, thereby improving the detection accuracy. Since there is no need to frequently move the ultrasonic probe 10, the detection efficiency is also improved. In other embodiments, as shown in FIG. 13, the field of view angle of each of the convex arrays 100 may also be a right angle. In another embodiment, as shown in FIG. 14, the field of view angle of each of the convex arrays 100 may also be an acute angle. The specific form can be set according to actual use requirements and is not limited here.

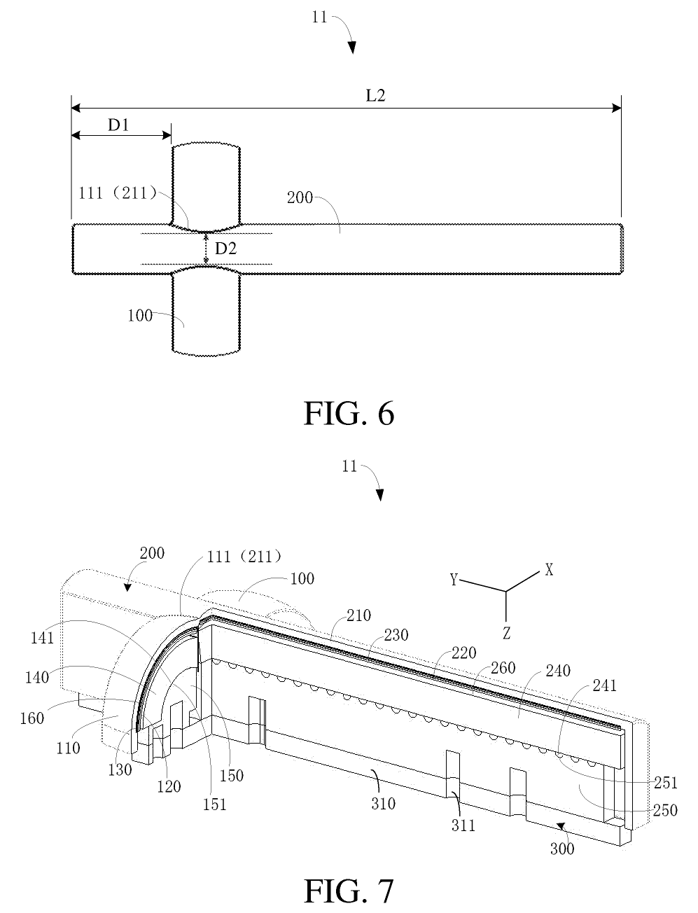

As shown in FIG. 6, in an embodiment, the linear array 200 extends a first distance D1 relative to the convex arrays 100 in a moving direction, and a ratio of the first distance D1 to the length L2 of the linear array 200 (i.e., the second length L2 of the second transducer 2) is within a range of 0 to 2/3 (excluding 0). In other words, a layout of the linear array 200 and the two convex arrays 100 is in a cross-shaped form. That is, the linear array 200 and the two convex arrays 100 are arranged to form a cross shape. Through this arrangement, it is ensured that the imaging plane P2 of the linear array 200 and the imaging plane P1 of the convex arrays 100 have a sufficient intersection area therebetween. Furthermore, the reliability of simultaneously observing the positions of the punctured tissue and the tip of the puncture needle through the intersection area is ensured, thereby improving the detection accuracy and detection efficiency. Preferably, the ratio of the first distance D1 that the linear array 200 extends relative to the convex arrays 100 to the length L2 of the linear array 200 is within a range of 0 to 1/2 (excluding 0). More preferably, the ratio of the first distance D1 that the linear array 200 extends relative to the convex arrays 100 to the length L2 of the linear array 200 is within a range of 1/4 to 1/2.

Referring to FIGS. 4 to 8, in an embodiment, each of the convex arrays 100 includes a first lens layer 110, and the first lens layer 110 has a first bonding surface 111. The linear array 200 includes a second lens layer 210, and the second lens layer 210 has a second bonding surface 211 connected to the first bonding surface 111. Ultrasonic beams emitted by the convex arrays 100 and the linear array 200 are focused by the first lens layers 110 and the second lens layer 210, respectively. The first bonding surface 111 and the second bonding surface 211 are connected to each other, thereby improving a sealing effect of the transducer assembly 11. Further, in some embodiments, the two first lens layers 110 and the second lens layer 210 between the two first lens layers 110 may be integrally formed to form an integral lens layer (e.g,) generally in a cross shape. In this way, the sealing surfaces to be assembled can be reduced, thereby reducing the hidden dangers caused by sealing failure. Meanwhile, the errors occurring during the assembly process can be reduced. In addition, a blind area of the coronal imaging area can be reduced, thereby increasing the accuracy of imaging detection. In some embodiments, the two first lens layers 110 may be integrated. In some embodiments, the two first lens layers 110 may also be manufactured separately from the second lens layer 210, and the two first lens layers 110 may be connected to the second lens layer 210 by means of bonding, etc. In some embodiments, the two first lens layers 110 may also be manufactured separately from the second lens layer 210, and the two first lens layers 110 may also be separated from the second lens layer 210 without bonding, which may be applicable to the later-described embodiments where the linear array 200 is slidable relative to the convex arrays 100, because it allows the two first lens layers 110 to move relative to the second lens layer 210.

As shown in FIGS. 4 to 8, in an embodiment, the two convex arrays 100 are symmetrically arranged on both sides of the linear array 200, so that the two first lens layers 110 are also symmetrically arranged on both sides of the linear array 200, and an orientation of each first bonding surface 111 is in conformity with an orientation of the second bonding surface 211 located on a same side. For example, the first bonding surface 111 and the second bonding surface 211 are each a cambered surface. In this way, the coronal imaging area of the convex arrays 100 and the sagittal imaging area of the linear array 200 can intersect with each other, i.e., there is an orthogonal plane between the coronal plane and the sagittal plane. Since the convex arrays 100 and the linear array 200 transmit and receive ultrasonic waves simultaneously during use, information of the orthogonal plane can be displayed on the same imaging plane. Therefore, during the puncture process, the doctor can simultaneously observe the positions of the punctured tissue and the tip of the puncture needle through the orthogonal plane, without the need for frequently moving the ultrasonic probe 10 to switch between the two planes, thereby accurately achieving the intracavitary puncture operation and improving the detection accuracy and detection efficiency. The first bonding surface 111 and the second bonding surface 211 may be bonded together by using an adhesive.

As shown in FIGS. 4 to 8, in another embodiment, in order to enhance the acoustic performance, a distance between the first bonding surfaces 111 of the two convex arrays 100 gradually decreases first and then gradually increases in the arrangement direction of the linear array 200, i.e., when viewed from the perspective of each of the convex arrays 100, its corresponding first bonding surface 111 is an outwardly convex cambered surface, so as to facilitate acoustic focusing. It can be understood that the second bonding surface 211 of the linear array 200 matches a shape of the first bonding surface 111 of the convex array 100. In this way, the transitions between the linear array 200 and the convex arrays 100 can be smoother, and it is not easy to hide dirt, and it is easy to clean.

As shown in FIG. 6, in an embodiment, a minimum distance D2 between the two first bonding surfaces 111 is greater than the elevation of the linear array 200, so as to ensure that the coronal plane imaged by the convex arrays and the sagittal plane imaged by the linear array have an intersection area therebetween, thereby ensuring the usage reliability of the ultrasonic probe 10.

As shown in FIGS. 7 to 9, in an embodiment, the two first lens layers 110 and the second lens layer 210 are in an integrated structure. By manufacturing the lens layers of the linear array 200 and the two convex arrays 100 in an integrated structure, the sealing surfaces to be assembled can be reduced, thereby reducing the hidden danger caused by sealing failure. Meanwhile, the errors occurring during the assembly process can be reduced. In addition, an imaging blind area can be also reduced, thereby increasing the accuracy of imaging detection.

As shown in FIGS. 7 to 9, in an embodiment, each of the convex arrays 100 further includes a first piezoelectric layer 120 stacked with the first lens layer 110 in the third direction. The linear array 200 further includes a second piezoelectric layer 220 stacked with the second lens layer 210 in the third direction. The first piezoelectric layer 120 and the second piezoelectric layer 220 are each configured to perform electroacoustic conversion, and are each a pipe part of the transducer. They convert an electrical signal excited by an ultrasonic system into ultrasonic waves. After entering the human body, the ultrasonic waves reflected at different tissue boundaries are converted into electrical signals by the piezoelectric layers and provided to the ultrasonic host for processing. The piezoelectric layer is the core component of the transducer. In the piezoelectric layer, piezoelectric array elements are formed, which have a one-to-one correspondence with the array elements in the aforementioned transducer. In other words, the arrangement of the piezoelectric array elements in the piezoelectric layer determines the arrangement of the array elements in the transducer where the piezoelectric layer is located. Therefore, in order to form the M array elements arranged in a row along a straight line extending in the second direction in the linear array 200 as described above, correspondingly, it is necessary to also form M piezoelectric array elements arranged in a row along a straight line extending in the second direction in the second piezoelectric layer 220 of the linear array 200, thereby forming a one-dimensional linear array. Similarly, in order to form the N array elements arranged in a row along an arc in a plane perpendicular to the second direction in the two convex arrays 100 as described above, correspondingly, it is necessary to also form N piezoelectric array elements arranged in a row along an arc in a plane perpendicular to the second direction in the two first piezoelectric layers 120, thereby forming the aforementioned combined one-dimensional convex array. As can be seen above, the arrangement of the piezoelectric array elements in the two first piezoelectric layers 120 may be different from the arrangement of the piezoelectric array elements in the second piezoelectric layer 220. Correspondingly, in the present disclosure, the two first piezoelectric layers 120 and the second piezoelectric layer 220 are manufactured separately to facilitate the formation of different arrangements of piezoelectric array elements. It should be noted that in the present disclosure, the two first piezoelectric layers 120 of the two convex arrays 100 are located on both sides of the linear array 200 and are separated by the linear array 200. Since the two first piezoelectric layers 120 and the second piezoelectric layer 220 are formed independently of each other, the two convex arrays 100 formed by the two first piezoelectric layers 120 and the linear array 200 formed by the second piezoelectric layer 220 can be allowed to operate independently of each other. For example, it is possible to allow only the linear array 200 to be turned on and the two convex arrays 100 to be turned off to use the linear array 200 for sagittal plane sensing; it is possible to allow only the two convex arrays 100 to be turned on and the linear array 200 to be turned off to use the two convex arrays 100 for coronal plane sensing; and it is also possible to simultaneously turn on both the linear array 200 and the two convex arrays 100 to perform sensing of both the sagittal plane and the coronal plane, thereby enabling simultaneous acquisition and display of information of the orthogonal plane between the two planes by sensing information of the two planes at the same time.

In an embodiment, the positive and negative surfaces of each of the first piezoelectric layers 120 and the second piezoelectric layer 220 are respectively coated with a conductive material to enhance the effect of the electroacoustic conversion.

In an embodiment, a bonding surface between the first piezoelectric layer 120 and the second piezoelectric layer 220 is a plane, and the first piezoelectric layer 120 and the second piezoelectric layer 220 may be bonded by using an adhesive. In another embodiment, the two first piezoelectric layers 120 may also be separated from the second piezoelectric layer 220 without bonding, which may be applicable to the later-described embodiments where the linear array 200 is slidable relative to the convex arrays 100, because it allows the two first piezoelectric layers 120 to move relative to the second piezoelectric layer 220.

As shown in FIGS. 7 to 9, in an embodiment, each of the convex arrays 100 further includes a first matching layer 130 stacked between the first lens layer 110 and the first piezoelectric layer 120. The linear array 200 further includes a second matching layer 230 stacked between the second lens layer 210 and the second piezoelectric layer 220. The first matching layer 130 and the second matching layer 230 are provided to cooperate with the first piezoelectric layer 120 and the second piezoelectric layer 220, respectively, so as to achieve acoustic matching. A bonding surface between the first matching layer 130 and the second matching layer 230 is a plane, and the first matching layer 130 and the second matching layer 230 may be bonded by using an adhesive. In another embodiment, the two first matching layers 130 may also be separated from the second matching layer 230 without bonding, which may be applicable to the later-described embodiments where the linear array 200 is slidable relative to the convex arrays 100, because it allows the two first matching layers 130 to move relative to the second matching layer 230. In some embodiments, the two first matching layers 130 may be spaced apart from each other and located on two sides of the second transducer 2.

As shown in FIGS. 7 to 9, in an embodiment, each of the convex arrays 100 further includes a first backing layer 140 stacked on a side of the first piezoelectric layer 120 facing away from the first matching layer 130. The linear array 200 further includes a second backing layer 240 stacked on a side of the second piezoelectric layer 220 facing away from the second matching layer 230. The first backing layer 140 and the second backing layer 240 are configured to support and absorb ultrasound for the first piezoelectric layer 120 and the second piezoelectric layer 220, respectively. The lens layers, the matching layers, and the backing layers may each be formed by casting. The lens layers may each be made of various materials suitable for making an acoustic lens, such as polyethylene synthetic resin, or the like. A bonding surface between the first backing layer 140 and the second backing layer 240 is a plane, and the first backing layer 140 and the second backing layer 240 may be bonded by using an adhesive.

As shown in FIGS. 7 to 9, in an embodiment, each of the convex arrays 100 further includes a first heat dissipation member 150 connected to the first backing layer 140. The linear array 200 further includes a second heat dissipation member 250 connected to the second backing layer 240. The first heat dissipation members 150 and the second heat dissipation member 250 are provided to dissipate heat from the ultrasonic probe 10, thereby reducing the possibility of discomfort to the human body caused by excessive temperature. The first heat dissipation members 150 and the second heat dissipation member 250 may each be made of a metal heat dissipation material such as aluminum alloy, copper alloy, or the like, or may be made of a heat-conductive non-metallic material such as graphene. In FIGS. 7 to 9, the two first heat dissipation members 150 and the second heat dissipation member 250 are each manufactured in a separate manner. However, in some other embodiments, some or all of the two first heat dissipation members 150 and the second heat dissipation member 250 may be manufactured in an integrated structure. For example, see FIG. 10, in the example of FIG. 10, the two first heat dissipation members 150 are manufactured in an integrated structure, while the second heat dissipation member 250 is manufactured in a separate manner from the first heat dissipation members 150 in the integrated structure. Wherein, the integrated structure formed by the two first heat dissipation members 150 may have a groove formed in the middle thereof, and the linear array 200 may be at least partially embedded in the groove. In this way, the sealing surfaces to be assembled can be reduced, and the errors occurring during the assembly process can also be reduced. Furthermore, in another embodiment, the two first heat dissipation members 150 and the second heat dissipation member 250 may be in an integrated structure to form a single cross-shaped heat dissipation member.

As shown in FIGS. 7 to 9, in an embodiment, each first heat dissipation member 150 is connected to a side of the corresponding first backing layer 140 facing away from the first matching layer 130. In other words, the first backing layer 140 and the first heat dissipation member 150 are arranged in the third direction (the Z direction in the figures). In this way, in addition to the heat dissipation and cooling effect, the first heat dissipation member 150 can also cooperate with the first backing layer 140 to further provide a good support effect for the first piezoelectric layer 120. In addition, by arranging the first heat dissipation member 150 below the first backing layer 140, a space occupied by the first heat dissipation member 150 and the first backing layer 140 in the first direction can be reduced, making the layout of the entire ultrasonic probe 10 more compact.

It can be understood that in other embodiments, the first heat dissipation member may be connected to a side of the first backing layer in the first direction; and the second heat dissipation member may be connected to a side of the second backing layer in the first direction.

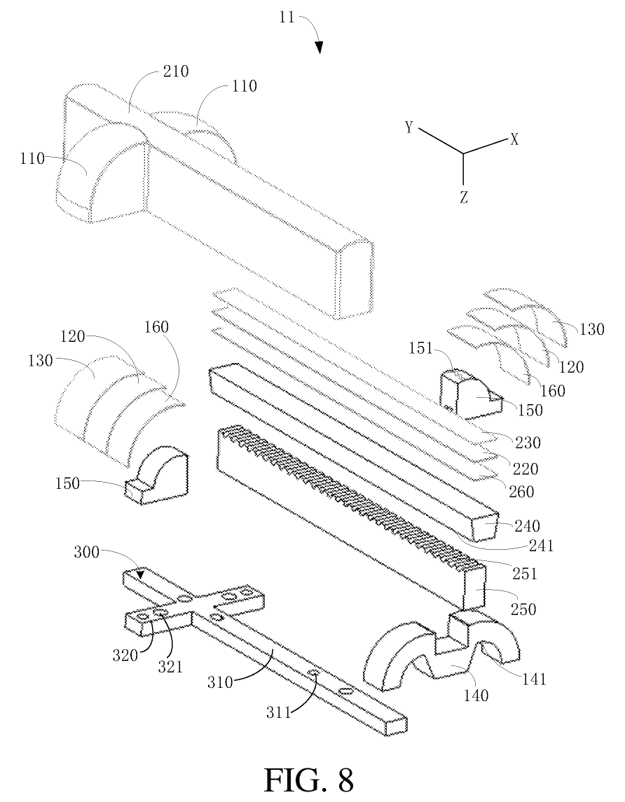

As shown in FIGS. 7 to 8, in an embodiment, the first backing layer 140 and the first heat dissipation member 150 are provided with a first concave-convex matching portion 141 and a second concave-convex matching portion 151 configured to engage with the first concave-convex matching portion 141, respectively. Taking the first heat dissipation member 150 as an example, surfaces between the first heat dissipation member 150 and the first backing layer 140 being in contact with each other are each designed as a concave-convex surface, so that after the first heat dissipation member 150 and the first backing layer 140 are stacked, the contact surfaces thereof are engaged with each other, i.e., a contact area between the first heat dissipation member 150 and the first backing layer 140 is relatively increased, so that it is not easy for the first heat dissipation member 150 and the first backing layer 140 to displace or slide relative to each other. In this way, not only a bonding effect between the first heat dissipation member 150 and the first backing layer 140 is increased, but also the reflection interface can be reduced, the reflection of the ultrasonic waves at a rear end of the ultrasonic probe 10 can be reduced, and the interferences can be reduced.

Specifically, the first concave-convex matching portion 141 provided on the first backing layer 140 is a clamping protrusion, and the second concave-convex matching portion 151 provided on the first heat dissipation member 150 is a clamping groove. Through the cooperation between the clamping protrusion and the clamping groove, the connection between the first backing layer 140 and the first heat dissipation member 150 is relatively tight, so that the first backing layer 140 and the first heat dissipation member 150 are not easily displaced relative to each other.

In another specific embodiment, the clamping protrusion and the clamping groove are each in a regular shape. For example, as shown in FIG. 8, a cross section of the clamping groove is triangular. Correspondingly, a shape of the clamping protrusion is adapted to a shape of the clamping groove, i.e., the shape of the clamping protrusion is a cone, and the cone can be a circular cone or a pyramid, so that the clamping protrusion and the clamping groove can be engaged with each other. In other embodiments, the shape of the clamping protrusion may be a cylinder, a cube or a cuboid. It can be understood that the shape of the clamping protrusion is not limited thereto, and the shape of the clamping protrusion may also be in an irregular shape.

Further, as shown in FIGS. 7 to 8, in another embodiment, the second backing layer 240 and the second heat dissipation member 250 are provided with a third concave-convex matching portion 241 and a fourth concave-convex matching portion 251 configured to engage with the third concave-convex matching portion 241, respectively. Through this arrangement, a connection effect between the second heat dissipation member 250 and the second backing layer 240 is increased. The second heat dissipation member 250 is connected to a side of the second backing layer 240 facing away from the second matching layer 230. Through this arrangement, after the second heat dissipation member 250 and the second backing layer 240 are stacked, the contact surfaces thereof are engaged with each other, i.e., a contact area between the second heat dissipation member 250 and the second backing layer 240 is relatively increased, so that it is not easy for the second heat dissipation member 250 and the second backing layer 240 to displace or slide relative to each other, thereby increasing a bonding effect between the second heat dissipation member 250 and the second backing layer 240. Moreover, the reflection interface can be reduced, the reflection of the ultrasonic waves at a rear end of the ultrasonic probe 10 can be reduced, and the interferences can be reduced.

It should be noted that, although in the examples shown in the figures of the present disclosure, the first heat dissipation members 150 and the second heat dissipation member 250 are illustratively shown as block-like structures and have the aforementioned second concave-convex matching portions 151 and fourth concave-convex matching portion 251 to facilitate engagement with the first backing layer 140 and the second backing layer 240, the shapes, structures, etc. of the first heat dissipation members 150 and the second heat dissipation member 250 may be appropriately changed as needed, as long as the expected heat dissipation function can be achieved. For example, in some embodiments, the first heat dissipation members 150 and the second heat dissipation member 250 may also be sheet-like heat dissipation materials respectively, and their surfaces facing the first backing layer 140 and the second backing layer 240 may also be smooth without the need to provide the second concave-convex matching portions 151 and the fourth concave-convex matching portion 251, and these sheet-like heat dissipation materials can be attached to the corresponding surfaces of the first backing layer 140 and the second backing layer 240 by bonding.

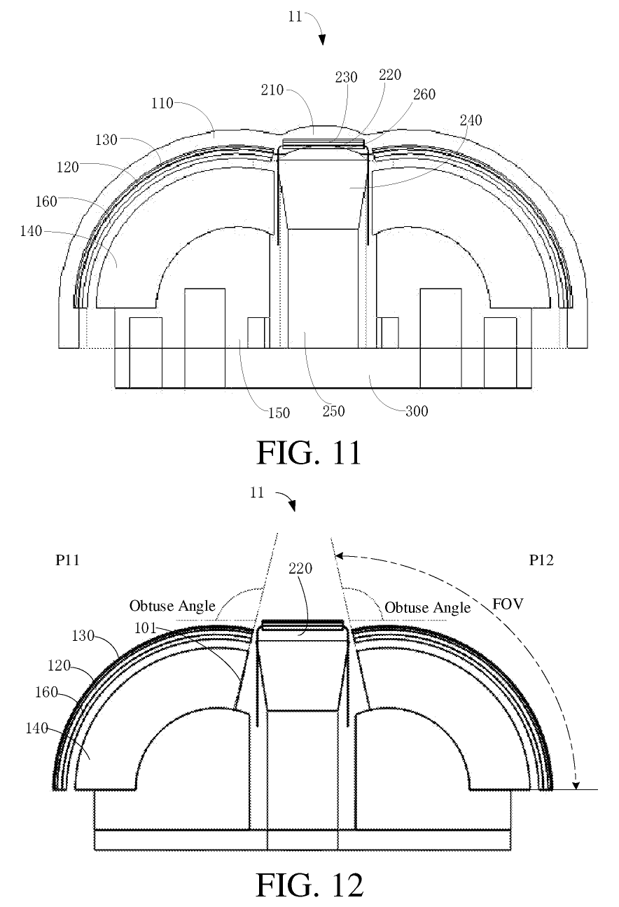

As shown in FIGS. 7 to 9, the two first backing layers 140 may be in an integrated structure. In some embodiments, the second backing layer 240 may be manufactured separately from the two first backing layers 140. In some embodiments, the two first backing layers 140 and the second backing layer 240 may be an integrated structure. By manufacturing the backing layers of the two convex arrays 100 in an integrated manner, the errors introduced during the assembly of the two convex arrays 100 are reduced, thereby ensuring that the two convex arrays 100 perform imaging on the same plane, so as to improve the accuracy of imaging. Wherein, the integrated structure formed by the two first backing layers 140 may have a groove formed in the middle thereof, and the linear array 200 may be at least partially embedded in the groove. As shown in FIG. 11, in another embodiment, the two first backing layers 140 may be manufactured in a separate manner. Furthermore, in another embodiment, the two first backing layers 140 and the second backing layer 240 may be in an integrated structure to form a single cross-shaped backing layer.

In some embodiments, as shown in FIGS. 7 to 9, in an embodiment, the first transducer 1 further includes a first flexible printed circuit layer 160 stacked between each first piezoelectric layer 120 and the corresponding first backing layer 140; the linear array 200 further includes a second flexible printed circuit layer 260 stacked between the second piezoelectric layer 220 and the second backing layer 240. The first flexible printed circuit layer 160 and the second flexible printed circuit layer 260 are respectively configured for electrical connection of the piezoelectric elements in the first piezoelectric layer 120 and the second piezoelectric layer 220, to achieve signal transmission between the piezoelectric elements in the first piezoelectric layer 120 and the second piezoelectric layer 220 and external components such as the mainboard 450. In the examples shown in FIGS. 7 to 9 of the present disclosure, the two first flexible printed circuit layers 160 and the second flexible printed circuit layer 260 are manufactured in a separate manner. A bonding surface between the first flexible printed circuit layer 160 and the second flexible printed circuit layer 260 is a plane, and the two may be bonded by using an adhesive. Alternatively, the two first flexible printed circuit layers 160 may also be separated from the second flexible printed circuit layer 260 without bonding, which may be applicable to the later-described embodiments where the linear array 200 is slidable relative to the two convex arrays 100, because it allows the two first flexible printed circuit layers 160 to move relative to the second flexible printed circuit layer 260. In another embodiment, the two first flexible printed circuit layers 160 may also be manufactured in an integrated structure with the second flexible printed circuit layer 260, to form an integral cross-shaped flexible printed circuit layer. Furthermore, the two first flexible printed circuit layers 160 may also be manufactured in an integrated structure, while the second flexible printed circuit layer 260 is manufactured separately. For example, the two first flexible printed circuit layers 160 may be connected to each other across the aforementioned intersection area I to form an integrated structure, and a groove may be formed in the middle of the integrated structure of the two first flexible printed circuit layers 160 (which can be formed by bending the first flexible printed circuit layers 160), and the linear array 200 may be at least partially embedded in the groove.

As shown in FIGS. 7 to 9, in an embodiment, the ultrasonic probe 10 further includes a connecting member 300, and the linear array 200 and the two convex arrays 100 are each connected to the connecting member 300. The linear array 200 and the two convex arrays 100 are positioned and fixed by the connecting member 300 to ensure the accuracy of positioning. In another embodiment, the ultrasonic probe 10 may not have the connecting member 300. Instead, the linear array 200 and the convex arrays 100 may be directly connected and positioned relative to each other, or the linear array 200 and the convex arrays 100 may each be connected to a housing so that the linear array 200 and the convex arrays 100 can be positioned and fixed by the housing.

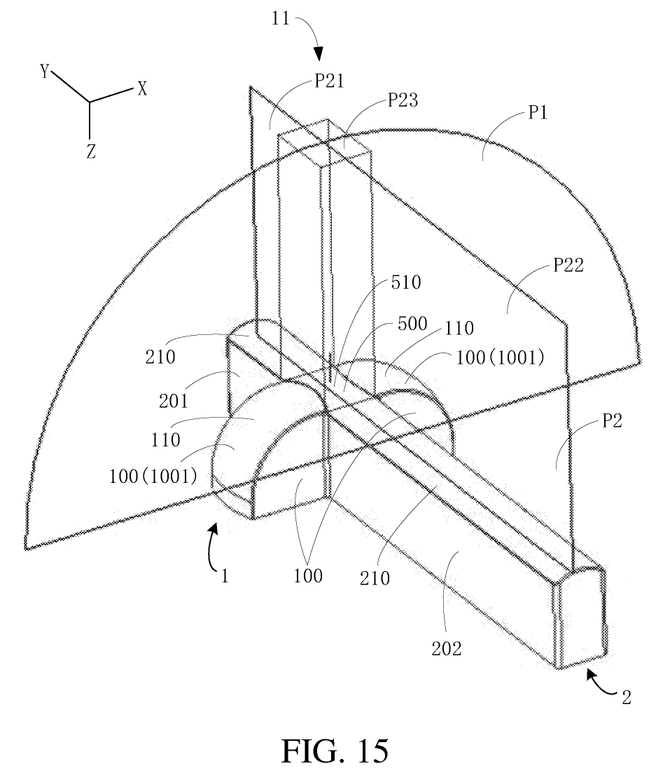

Further, as shown in FIG. 8, the connecting member 300 includes a first support section 310 and second support sections 320 connected to both sides of the first support section 310 in the first direction, i.e., the connecting member 300 is a cross-shaped sheet structure, so as to be compatible with the arrangement positions of the convex arrays 100 and the linear array 200. The first support section 310 and the second support sections 320 may be manufactured in a separate manner or manufactured in an integrated structure. The ultrasonic probe 10 further includes a first fastener and second fasteners. The first fastener is configured to connect the first support section 310 and the linear array 200, and each second fastener is configured to connect the second support section 320 and the convex array 100 located on a same side. Specifically, as shown in FIGS. 7 to 9, the first support section 310 and the second heat dissipation member 250 are each provided with a first screw hole 311, and the first fastener is inserted through the first screw holes 311 of the first support section 310 and the second heat dissipation member 250 to achieve the connection between the first support section 310 and the second heat dissipation member 250. The second support section 320 and the first heat dissipation member 150 are each provided with a second screw hole 321, and the second fastener is inserted through the second screw holes 321 of the second support section 320 and the first heat dissipation member 150 to achieve the connection between the second support section 320 and the first heat dissipation member 150. In this way, the connection effect between the components is improved, thereby improving the usage reliability of the ultrasonic probe 10. The first fastener and the second fasteners may be screws, etc. In other embodiments, the first heat dissipation members and the second heat dissipation member may also be bonded to the connecting member by using an adhesive. Furthermore, in some embodiments, the two first heat dissipation members 150 may be manufactured in an integrated structure with the connecting member 300, and then the second heat dissipation member 250 may be connected to this integrated structure through the above fasteners or by bonding. For such an example, see, for example, FIGS. 15 and 17. In some implementations, the second heat dissipation member 250 may be manufactured in an integrated structure with the connecting member 300, and then the two first heat dissipation members 150 may be connected to this integrated structure through the above fasteners or by bonding. In some embodiments, the two first heat dissipation members 150 and the second heat dissipation member 250 may be combined with the connecting member 300 into an integrated structure, and the remaining components in the two convex arrays 100 and the linear array 200 may be fixed to this integrated structure, so that this integrated structure serves both for heat dissipation and as a connecting member to connect the two convex arrays 100 and the linear array 200 together.

In still some embodiments, the second support sections 320 are slidably connected to the first support section 310, and can drive the two convex arrays 100 to move in the arrangement direction of the linear array 200. In this way, the distance of the linear array 200 extending relative to the convex arrays 100 can be conveniently adjusted, thereby meeting actual use requirements and being more flexible in use.

As can be seen above, in the various layer structures of the various embodiments of the present disclosure described above, for the first lens layer 110, the first matching layer 130, and the first piezoelectric layer 120 located on the left side, and the first lens layer 110, the first matching layer 130, and the first piezoelectric layer 120 located on the right side, each pair of layers with the same function in these layer structures is separately arranged on the left and right sides of the linear array 200 and is spaced apart by the linear array 200. These layer structures can determine the field of view of the convex arrays 100. In this way, the left convex array 100 and the right convex array 100 can perform sensing on the left and right sides of the linear array 200 to form roughly fan-shaped imaging areas P11 and P12, respectively. In some alternative embodiments, the first lens layers 110 may be integrated with the second lens layer 210. In addition to the above various layers, the remaining layers (or components) in the two convex arrays 100, including the first flexible printed circuit layer 160, the first backing layer 140, and the first heat dissipation member 150, can either be separately arranged on the left and right sides of the linear array 200, or the corresponding layers (or components) on the left and right sides can bypass below the linear array 200 and span the aforementioned intersection area I to form an integrated structure. For details, please refer to the descriptions in the above embodiments.

The transducer assembly 11 of the ultrasonic probe 10 according to the first embodiment includes one linear array and two convex arrays at least partially arranged on both sides of the linear array. An inner wall of a cavity and a tissue close thereto are detected by the convex arrays to obtain a coronal plane image, so that a position of a punctured tissue can be observed. A position of a puncture needle can be detected by the linear array to obtain a sagittal plane image, so that the position of the tip of the puncture needle can be observed. The convex arrays and the linear array transmit and receive simultaneously during use, so that the positions of the punctured tissue and the tip of the puncture needle can be observed simultaneously, without the need for frequently moving the ultrasonic probe to switch between two planes, thereby accurately achieving the intracavitary puncture operations and improving the detection accuracy and efficiency.

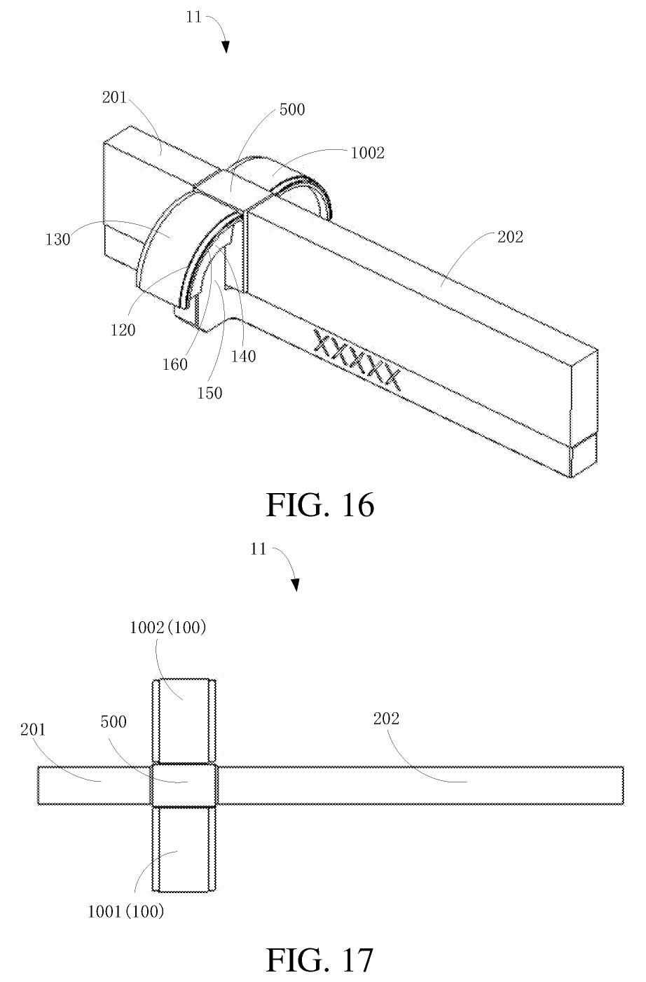

Referring to FIGS. 15 to 18, a transducer assembly 11 of the ultrasonic probe 10 according to a second embodiment of the present disclosure is provided. The main difference between the transducer assembly 11 of this second embodiment and the transducer assembly 11 of the first embodiment lies in that the second transducer 2 does not include a whole linear array 200 arranged in the two second sub-areas s2 and the intersection area I, but instead, a first area array 500 is arranged in the intersection area I, and correspondingly, a first sub-linear array 201 and a second sub-linear array 202 are respectively arranged in the two second sub-areas s2. For the sake of brevity, in the following description, the parts different from the first embodiment will be mainly described. Apart from that, unless there is a clear conflict, the structures and configurations of the remaining parts in this second embodiment that are similar to those in the first embodiment can be referred to the descriptions in the first embodiment, and will not be repeated here.

Similar to the first embodiment, in this second embodiment, the second transducer 2 is arranged in the two second sub-areas s2 and the intersection area I. The first transducer 1 is at least partially arranged in the two second sub-areas s2, and at least a part of components in the first transducer 1 are separated by the second transducer 2. In addition, optionally, some components in the first transducer 1 may also extend into the intersection area I.

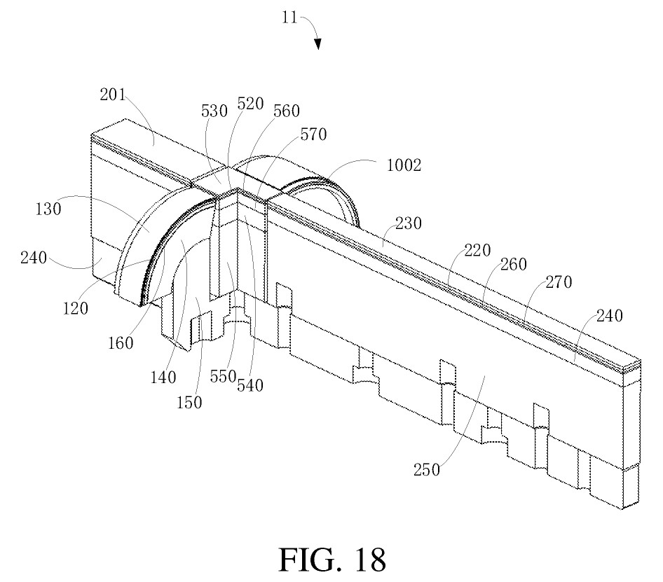

Referring to FIGS. 15 to 18, the first transducer 1 in this second embodiment may have substantially the same configuration as the first transducer 1 in the first embodiment. In this second embodiment, the first transducer 1 includes two convex arrays 100 arranged in the first direction. The left convex array 100 may include the first portion 1001, and the right convex array 100 may include the second portion 1002. The linear array 200 is connected between the first portion 1001 and the second portion 1002. Referring to FIG. 18, in the direction from top to bottom, each convex array 100 in this embodiment may also sequentially include a first lens layer 110, a first matching layer 130, a first piezoelectric layer 120, a first flexible printed circuit layer 160, a first backing layer 140, and a first heat dissipation member 150. The structures, functions, relationships with other components, etc. of the various components in the linear array 200 in this second embodiment can be similarly referred to the descriptions in the first embodiment, and will not be repeated here.

In this second embodiment, the second transducer 2 is arranged in the second direction, and in the second direction, respectively includes a first sub-linear array 201 arranged in the front second sub-area s2, a first area array 500 arranged in the intersection area I, and a second sub-linear array 202 arranged in the rear second sub-area s2, and the first area array 500 is connected between the first sub-linear array 201 and the second sub-linear array 202. Wherein, the first sub-linear array 201 and the second sub-linear array 202 in the two second sub-areas s2 may respectively have substantially the same configuration as the two corresponding parts of the linear array 200 in the first embodiment located in the two second sub-areas s2. Specifically, referring to FIG. 18, in the direction from top to bottom, each of the first sub-linear array 201 and the second sub-linear array 202 in this embodiment may also sequentially include a second lens layer 210, a second matching layer 230, a second piezoelectric layer 220, a second flexible printed circuit layer 260, a second backing layer 240, and a second heat dissipation member 250. Wherein, the respective layers in the first sub-linear array 201 may be aligned one-to-one with the respective layers in the second sub-linear array 202, but are separated by the first area array 500, so as to have the same configuration as the layers in the same position parts of the linear array 200 in the first embodiment, and such that the imaging areas of the first sub-linear array 201 and the second sub-linear array 202 both fall within the same sagittal plane imaging plane P2. For instance, the one-dimensional linear array formed by the array elements in the first sub-linear array 201 and the one-dimensional linear array formed by the array elements in the second sub-linear array 202 may be located on the same straight line extending in the second direction. The structures, functions, relationships with other components, etc. of the various components in the first sub-linear array 201 and the second sub-linear array 202 in this second embodiment can be similarly referred to the descriptions of the two corresponding parts of the linear array 200 in the first embodiment located in the two second sub-areas s2, and will not be repeated here.

The first area array 500 may also include a plurality of array elements. Different from the linear array and the convex array formed as one-dimensional arrays, the plurality of array elements in the first area array 500 can be arranged in a matrix on a plane to form a two-dimensional array (2D Array), and the two-dimensional array can scan at the same time to obtain a three-dimensional image within a three-dimensional volume enclosed by the plane where the two-dimensional array is located and the detection directions of the array elements at the outermost periphery of the two-dimensional array. Specifically, in this embodiment, the plurality of array elements of the first area array 500 can be arranged in a matrix in a rectangular plane within the intersection area I and perpendicular to the third direction, for example, can be arranged in m rows along the first direction and n columns along the second direction to form an m*n two-dimensional area array (2D area array).

Referring to FIG. 18, in the direction from top to bottom, the first area array 500 in this embodiment may sequentially include a third lens layer 510, a third matching layer 530, a third piezoelectric layer 520, a third flexible printed circuit layer 560, a chip layer 570, a third backing layer 540, and a third heat dissipation member 550. The third lens layer 510 may be formed integrally with the first lens layers 110 of the convex arrays 100 and the second lens layers 210 of the first sub-linear array 201 and the second sub-linear array 202, or may be manufactured separately. Wherein, in order to form the m*n two-dimensional area array in the first area array 500 as described above, correspondingly, it is necessary to also form m*n piezoelectric array elements arranged in a matrix in a rectangular plane within the intersection area I and perpendicular to the third direction in the third piezoelectric layer 520 of the first area array 500.

In this embodiment, similar to the first embodiment, the two convex arrays 100 are configured to detect the two-dimensional coronal plane imaging plane P1, wherein the left convex array 100 forms a roughly fan-shaped two-dimensional imaging area P11, and the right convex array 100 forms a roughly fan-shaped two-dimensional imaging area P12, and the imaging areas P11 and P12 are both within the coronal plane imaging plane P1. The first sub-linear array 201 may have a rectangular two-dimensional imaging area P21 located above the one-dimensional linear array in the first sub-linear array 201, and the second sub-linear array 202 may have a rectangular two-dimensional imaging area P22 located above the one-dimensional linear array in the second sub-linear array 202, and the imaging areas P21 and P22 are both within the sagittal plane imaging plane P2. In the above arrangement, referring to FIGS. 12, 13, and 14, since at least the upper half portion of the left convex array 100 and at least the upper half portion of the right convex array 100 are separated by the second transducer 2, in the area above the intersection area I close to the upper surface of the second transducer 2, there may be an area (blind area) between the imaging areas P11 and P12 that cannot be detected by the left and right convex arrays 100. Furthermore, referring to FIG. 15, since the part of the linear array 200 located in the intersection area I is replaced by the first area array 500, above the intersection area I, there is also an area (blind area) between the imaging areas P21 and P22 that cannot be detected by the first sub-linear array 201 and the second sub-linear array 202. In this second embodiment, by arranging the first area array 500 in the intersection area I, the array elements in the first area array 500 form a two-dimensional area array arranged in a matrix along both the first direction and the second direction, and the imaging area P23 of the first area array 500 is a three-dimensional rectangular columnar area located above the two-dimensional area array in the first area array 500, thereby being able to simultaneously detect information of the two planes, the coronal plane imaging plane P1 and the sagittal plane imaging plane P2, above the intersection area I, and simultaneously compensate for the above blind areas of the convex arrays 100 in the first transducer 1 and the linear array 200 in the second transducer 2, ensuring that the transducer assembly 11 can simultaneously detect the information of the orthogonal plane between the coronal plane and the sagittal plane in the intersection area I.

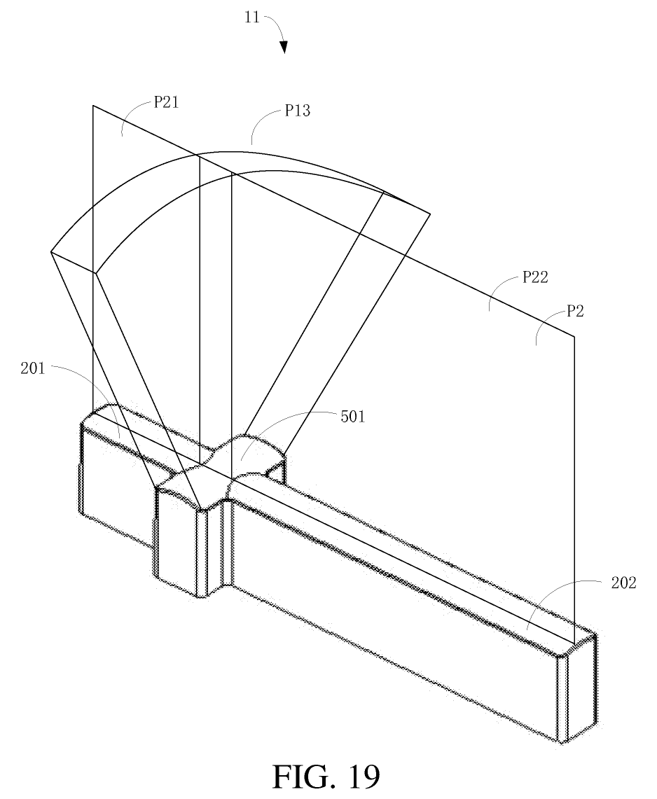

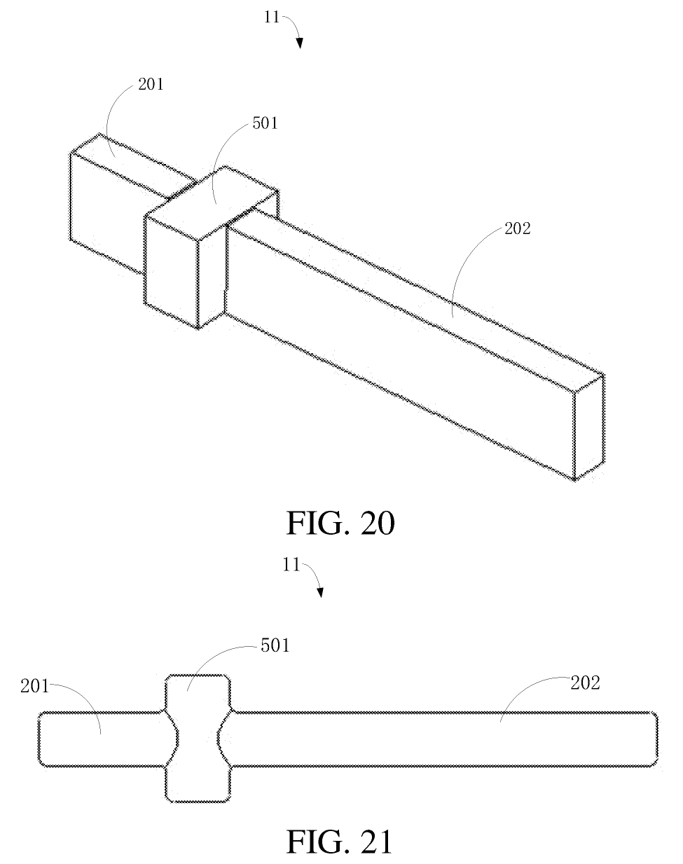

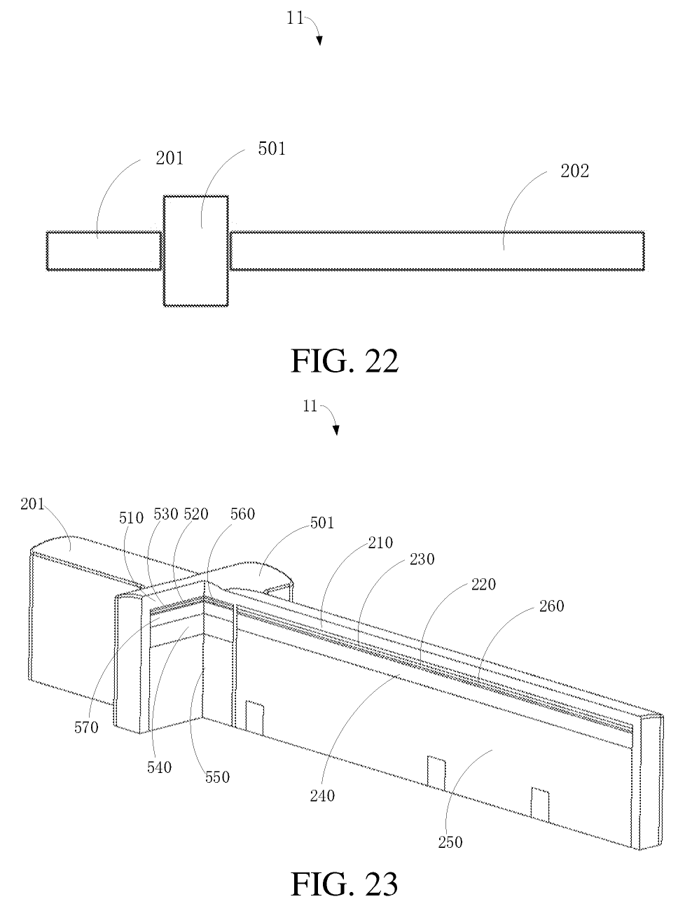

Referring to FIGS. 19 to 23, a transducer assembly 11 of the ultrasonic probe 10 according to a third embodiment of the present disclosure is provided. The main difference between the transducer assembly 11 of this third embodiment and the transducer assembly 11 of the second embodiment lies in that the first transducer 1 does not include the convex arrays 100, but includes a whole second area array 501 arranged in the two first sub-areas s1 and the intersection area I. The second transducer 2 only includes the first sub-linear array 201 and the second sub-linear array 202 respectively arranged in the two second sub-areas s2, and does not include the first area array 500. For the sake of brevity, in the following description, the parts different from the second embodiment will be mainly described. Apart from that, unless there is a clear conflict, the structures and configurations of the remaining parts in this third embodiment that are similar to those in the second embodiment (and thus also indirectly to those in the first embodiment) can be referred to the descriptions in the second embodiment, and will not be repeated here.

In this third embodiment, the first transducer 1 is arranged in the two first sub-areas s1 and the intersection area I, and the second transducer 2 is arranged in the two second sub-areas s2.

Referring to FIGS. 19 to 23, the first transducer 1 includes a whole second area array 501 arranged in the two first sub-areas s1 and the intersection area I. The second transducer 2 includes a first sub-linear array 201 arranged in the front second sub-area s2 and a second sub-linear array 202 arranged in the rear second sub-area s2. Wherein, the first sub-linear array 201 and the second sub-linear array 202 in the two second sub-areas s2 may respectively have substantially the same configuration as the first sub-linear array 201 and the second sub-linear array 202 in the two second sub-areas s2 in the second embodiment, and thus can be similarly referred to the descriptions in the second embodiment, and will not be repeated here. Except that the second area array 501 has a larger arrangement area than the first area array 500 in the second embodiment, its various layer structures can be similar to those of the first area array 500 in the second embodiment. For example, referring to FIG. 23, in the direction from top to bottom, the second area array 501 may also sequentially include a third lens layer 510, a third matching layer 530, a third piezoelectric layer 520, a third flexible printed circuit layer 560, a chip layer 570, a third backing layer 540, and a third heat dissipation member 550, and thus can be similarly referred to the descriptions in the second embodiment, and will not be repeated here.

Similarly, referring to FIG. 19, the first sub-linear array 201 may have a rectangular two-dimensional imaging area P21 located above the one-dimensional linear array in the first sub-linear array 201, and the second sub-linear array 202 may have a rectangular two-dimensional imaging area P22 located above the one-dimensional linear array in the second sub-linear array 202, and the imaging areas P21 and P22 are both within the sagittal plane imaging plane P2. Different from the first area array 500, through known techniques such as electronic delay focusing and beam angle deflection, the field of view angle of the second area array 501 can be broadened, so that the second area array 501 can detect information within a fan-shaped imaging area above it. Correspondingly, referring to FIG. 19, the imaging area P24 of the second area array 501 may be the three-dimensional block-shaped imaging area P13 with a fan-shaped cross-section located above the two-dimensional area array in the second area array 501. In this way, this fan-shaped three-dimensional block-shaped imaging area P13 can also include the information of the aforementioned coronal plane imaging plane P1. Thereby, while detecting the coronal plane imaging plane P1 above the two first sub-areas s1, the information of the two planes, the coronal plane imaging plane P1 and the sagittal plane imaging plane P2, above the intersection area I can also be simultaneously detected, ensuring that the transducer assembly 11 can simultaneously detect the information of the orthogonal plane between the coronal plane and the sagittal plane in the intersection area I.

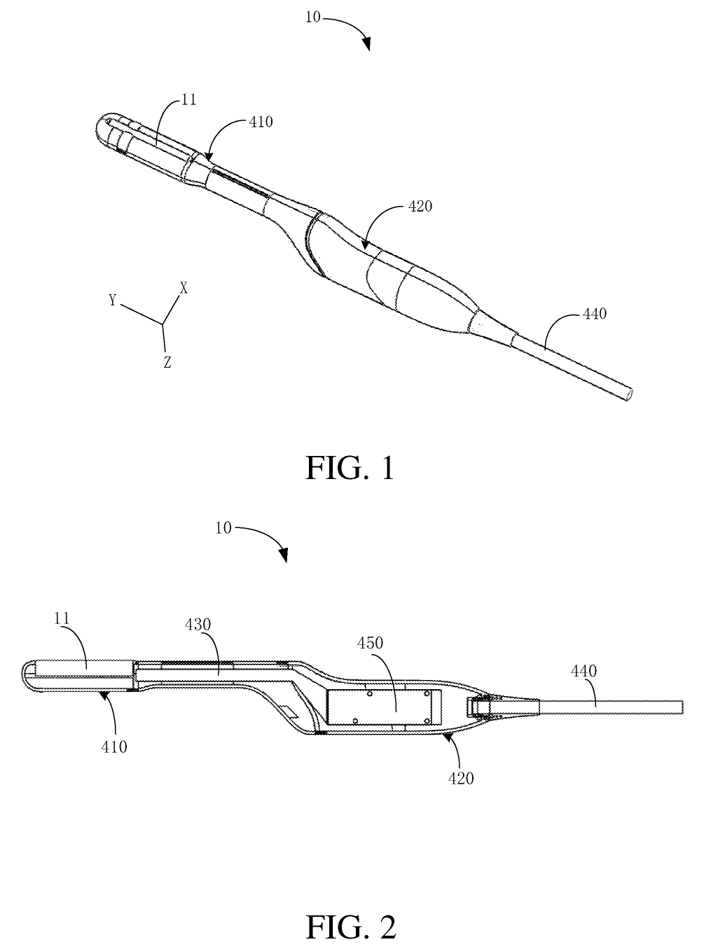

As shown in FIGS. 1 to 2, in some embodiments, the ultrasonic probe 10 further includes a shell acoustic head end 410, a shell handle end 420, an electrical connection lead 430, a mainboard 450, and a cable 440. The linear array 200 and the convex arrays 100 in the transducer assembly 11 are integrated in the shell acoustic head end 410, and the mainboard 450 is located at the shell handle end 420. The electrical connection lead 430 is configured to achieve an electrical connection between the transducer assembly 11 and the mainboard 450. The cable 440 is configured to connect to the host of the ultrasonic device, so as to realize the input and output of signals. Of course, it is not limited thereto, and the ultrasonic probe 10 may also be connected to the host in a wireless manner. During the process of inserting the shell acoustic head end 410 into the cavity, in order to improve the comfort of examination and alleviate the pain of the patient, an outer shape of the shell acoustic head end 410 may be configured to be cylindrical, and a transition area where a diameter change is designed to be arc-shaped to form a smooth transition. Since there is no sharp structure such as edges and corners on an outer surface of the ultrasonic probe 10, the patient feels more comfortable during the insertion process. Parameters such as the length and diameter of the shell acoustic head end 410 may be set to match the cavity to be examined.

Furthermore, an embodiment of the present disclosure further provides an ultrasonic device (not shown), including a host (not shown), a display (not shown), and an ultrasonic probe 10 according to any of the above embodiments. The ultrasonic probe 10 is configured to acquire ultrasonic imaging data. The host is communicatively connected to the ultrasonic probe 10, and is configured to receive and process the ultrasonic imaging data to generate an ultrasonic image. The display is connected to the host, and is configured to display the ultrasonic image.

Taking the application of the ultrasonic probe in intracavitary ultrasound puncture diagnosis as an example, since the ultrasonic device includes the ultrasonic probe of the embodiment as described above, the doctor can simultaneously observe the positions of the punctured tissue and the tip of the puncture needle through the orthogonal plane during the puncture process, without the need for frequently moving the ultrasonic probe to switch between the two planes, thereby accurately achieving the intracavitary puncture operation and improving the detection accuracy and efficiency.

In addition to the ultrasonic probe, the host and the display device described above, the ultrasonic device of the embodiment of the present disclosure may further include other components, such as a trolley, and these related components can refer to the prior art.

The technical features in the above embodiments may be combined arbitrarily. For concise description, not all possible combinations of the technical features in the above embodiments are described. However, provided that they do not conflict with each other, all combinations of the technical features are to be considered to be within the scope described in this specification.

The above-mentioned embodiments only describe several implementations of the present disclosure, and their description is specific and detailed, but should not be understood as a limitation on the patent scope of the present disclosure. It should be noted that, for a person of ordinary skill in the art may further make variations and improvements without departing from the conception of the present disclosure, and these all fall within the protection scope of the present disclosure. Therefore, the protection scope of the present disclosure should be subject to the appended claims.

What is claimed is:

1. An ultrasonic probe, comprising a transducer assembly, wherein the transducer assembly comprises:
a first transducer arranged in a first direction and at least including a first portion and a second portion; and
a second transducer arranged in a second direction, the second direction being perpendicular to the first direction;
wherein the second transducer is located between the first portion and the second portion of the first transducer.

2. The ultrasonic probe according to claim 1, wherein the first transducer comprises two convex arrays arranged in the first direction, the first portion of the first transducer includes one of the two convex arrays located on a first side in the first direction, and the second portion of the first transducer includes the other of the two convex arrays located on a second side opposite to the first side in the first direction; and
the second transducer comprises a linear array arranged in the second direction, and the linear array is arranged between the first portion and the second portion.

3. The ultrasonic probe according to claim 1, wherein the first portion of the first transducer and the second portion of the first transducer are two independent convex arrays, respectively.

4. The ultrasonic probe according to claim 1, wherein the first portion of the first transducer and the second portion of the first transducer are integrated.

5. The ultrasonic probe according to claim 1, wherein each of the first portion of the first transducer and the second portion of the first transducer comprises a first lens layer, and, the linear array comprises a second lens layer.

6. The ultrasonic probe according to claim 5, the second lens layer and two first lens layers included in the first portion of the first transducer and the second portion of the first transducer are integrated.

7. The ultrasonic probe according to claim 5, wherein each of the first portion of the first transducer and the second portion of the first transducer further comprises a first piezoelectric layer stacked with the first lens layer in a third direction, the linear array further comprises a second piezoelectric layer stacked with the second lens layer in the third direction, two first piezoelectric layers included in the first portion of the first transducer and the second portion of the first transducer and the second piezoelectric layer are space apart from each other and located on two sides of the second transducer in the first direction.

8. The ultrasonic probe according to claim 5, wherein each of the first portion of the first transducer and the second portion of the first transducer further comprises a first piezoelectric layer stacked with the first lens layer in a third direction;
each of the first portion of the first transducer and the second portion of the first transducer further comprises a first matching layer arranged between the first lens layer and the first piezoelectric layer;
each of the first portion of the first transducer and the second portion of the first transducer further comprises a first backing layer stacked on a side of the first piezoelectric layer facing away from the first matching layer;
the linear array further comprises a second piezoelectric layer stacked with the second lens layer in the third direction;
the linear array further comprises a second matching layer arranged between the second lens layer and the second piezoelectric layer; and
the linear array further comprises a second backing layer stacked on a side of the second piezoelectric layer facing away from the second matching layer;
wherein the third direction, the first direction, and the second direction are perpendicular to each other.

9. The ultrasonic probe according to claim 8, wherein each of the first portion of the first transducer and the second portion of the first transducer further comprises a first heat dissipation member connected to the first backing layer; and
the linear array further comprises a second heat dissipation member connected to the second backing layer.

10. The ultrasonic probe according to claim 9, wherein the first backing layer and the first heat dissipation member are provided with a first concave-convex matching portion and a second concave-convex matching portion configured to engage with the first concave-convex matching portion, respectively; and/or
the second backing layer and the second heat dissipation member are provided with a third concave-convex matching portion and a fourth concave-convex matching portion configured to engage with the third concave-convex matching portion, respectively.

11. The ultrasonic probe according to claim 10, wherein the first heat dissipation member is connected to a side of the first backing layer facing away from the first matching layer; and/or
the second heat dissipation member is connected to a side of the second backing layer facing away from the second matching layer.

12. The ultrasonic probe according to claim 9, wherein two first backing layers included in each of the first portion of the first transducer and the second portion of the first transducer are in an integrated structure.

13. The ultrasonic probe according to claim 9, wherein two first heat dissipation members included in each of the first portion of the first transducer and the second portion of the first transducer are in an integrated structure.

14. The ultrasonic probe according to claim 1, wherein the ultrasonic probe further comprises a connecting member, and the linear array, the first portion of the first transducer, and the second portion of the first transducer are each connected to the connecting member.

15. The ultrasonic probe according to claim 14, wherein the connecting member comprises a first support section and second support sections connected to both sides of the first support section in the first direction;
the ultrasonic probe further comprises a first fastener configured to connect the first support section and the linear array; and
the ultrasonic probe further comprises second fasteners, each of the second fasteners being configured to connect each of the second support sections and one of the convex arrays located on a same side.

16. The ultrasonic probe according to claim 15, wherein the second support sections are slidably connected to the first support section, and are capable of driving the convex arrays to move in the second direction.

17. The ultrasonic probe according to claim 1, wherein the first transducer comprises two convex arrays arranged in the first direction, the first portion includes one of the two convex arrays located on a first side in the first direction, and the second portion includes the other of the two convex arrays located on a second side opposite to the first side in the first direction; and
the second transducer comprises a first sub-linear array, a first area array, and a second sub-linear array arranged in the second direction, the first area array is connected between the first sub-linear array and the second sub-linear array, and the first area array is arranged between the first portion and the second portion.

18. An ultrasonic probe, comprising a transducer assembly, wherein the transducer assembly comprises:
a first transducer arranged in a first direction and at least including a first portion and a second portion; and
a second transducer arranged in a second direction, the second direction being perpendicular to the first direction;
wherein the second transducer is located between the first portion and the second portion of the first transducer, and wherein, in the first direction, a first piezoelectric layer in the first portion of the first transducer and a first piezoelectric layer in the second portion of the first transducer are spaced apart from each other and located on two sides of the second transducer.

19. An ultrasonic probe, comprising a transducer assembly, wherein the transducer assembly comprises:
a first transducer comprising a second area array arranged in a first direction; and
a second transducer comprising a first sub-linear array and a second sub-linear array arranged in a second direction, the second direction being perpendicular to the first direction;
wherein the second area array is located between the first sub-linear array and the second sub-linear array.

20. An ultrasonic device, comprising:
an ultrasonic probe according to the ultrasonic probe of claim 1, the ultrasonic probe being configured to acquire ultrasonic imaging data;
a host communicatively connected with the ultrasonic probe, and configured to receive and process the ultrasonic imaging data to generate an ultrasonic image; and
a display connected to the host, and configured to display the ultrasonic image.
